(19) United States
(12) Patent Application Publication
(10) Pub. No.: US 2009/0226955 A1
(43) Pub. Date: Sep. 10, 2009

(54) Immortalized retinal pigmented epithelial cells

(75) Inventors: Sharon J. Elliot, Miami, FL (US); Paola Catanuto, Miami, FL (US); Scott W. Cousins, Chapel Hill, NC (US)

Correspondence Address: VENABLE LLP, P.O. BOX 34385, WASHINGTON, DC, 20043-9998, US

(73) Assignee: University of Miami, Miami, FL (US)

(21) Appl. No.: 12/318,152

(22) Filed: Dec. 22, 2008

Related U.S. Application Data
(60) Provisional application No. 61/008,575, filed on Dec. 21, 2007.

(51) Int. Cl. C12Q 1/02; C12N 5/00; C12N 5/02; C12N 5/06; C12N 5/08

(52) U.S. Cl. 435/29; 435/325; 435/375; 435/354; 435/366

(57) ABSTRACT

The present invention provides isolated cultures of immortalized retinal pigmented epithelial cells. The cells retain one or more of the in vivo characteristics of non-immortalized retinal pigmented epithelial cells, including, for example, cobblestone morphology, polarization, the presence and/or expression of one or more protein markers, and a metabolic pattern typical of non-immortalized retinal pigmented epithelial cells. The present invention also provides methods of making and using immortalized retinal pigmented epithelial cells.

This application claims priority to U.S. Provisional Application No. 61/008,575, filed Dec. 21, 2007, which is hereby incorporated by reference in its entirety and for all purposes.

Research described in this application was supported by grant RO1 EY014477 from the National Eye Institute, a part of the National Institutes of Health. The government may have certain rights in this application.

### FIELD OF THE INVENTION

The invention relates to isolated cultures of immortalized retinal pigmented epithelial cells, and methods of making and using the cultures.

### BACKGROUND

The study of in vivo pathology has been greatly advanced through the utilization of isolated cells from the tissue of interest. In particular, the complexity of the intact eye has made ocular cell culture an attractive tool for researchers studying eye disease. To date many studies on human retinal diseases have utilized the human retinal pigmented epithelial (RPE) cell line ARPE-19 or other cell lines that proliferate for a finite number of passages. These cells retain their differentiated characteristics in vitro, but the difficulty of obtaining human eyes suitable for cell culture, and the physiologic differences between donors, make studies with human material difficult at best.

There is increasing interest in mouse models of ocular disease, including age related macular degeneration (“AMD”). The ability to sustain cultures of mouse RPE cells derived from these models would be highly advantageous. To date no one has immortalized mouse RPE cells. There remains a need for isolated immortalized RPE cell lines, including those that retain their cobblestone morphology as well as other in vivo characteristics of RPE cells, such as polarization and the presence of protein markers.

### SUMMARY

The present invention meets the need in the art for immortalized retinal pigmented epithelial cell lines that retain their in vivo phenotype. These cell lines can aid in studies of ocular diseases such as age related macular degeneration.

In one aspect, the invention provides isolated cultures of immortalized RPE cells. In some embodiments, each immortalized cell comprises one or more genes that confer immortality. The genes can be provided by a vector such as a viral vector and can comprise, for example, genes E6 and/or E7 from HPV 16. The genes can also include, for example, telomerase reverse transcriptase genes. The immortalized cells retain one or more of the in vivo characteristics of non-immortalized RPE cells, such as, without limitation, cobblestone morphology, polarization, a metabolic pattern typical of non-immortalized RPE cells, and/or one or more protein markers including retinal pigment epithelium-specific protein 65 kDa (“RPE65”), cellular retinaldehyde binding protein (CRALBP), matrix metalloproteinase 2 (“MMP-2”), tissue inhibitor of metalloproteinase 2 (“TIMP-2”), collagen type IV, estrogen receptor (“ER”)α, ERβ, zona occludens-1 (“ZO1”), cytokeratin 8, cytokeratin 18, ER copy number ratio, and combinations thereof. In some embodiments, the protein markers are expressed in substantially the same amounts as in RPE cells. The cells to be immortalized can be harvested from a subject, such as, for example, a mouse or a human.

In some embodiments, the immortalized cells can exhibit one or more characteristics of an ocular disease, for example AMD. The cells may have exhibited the characteristics of the ocular disease prior to harvesting from the subject, or they may be modified after harvesting so as to exhibit the one or more characteristics of an ocular disease. This modification can take place before, simultaneously with, or after immortalization.

In another aspect, the invention provides methods of preparing an immortalized RPE cell line. The methods can comprise the steps of, for example, harvesting RPE cells from a subject, and culturing the RPE cells in a medium under conditions such that the cells are immortalized. The method can further comprise combining the RPE cells with a viral vector comprising a gene that confers resistance to an antibiotic such as, for example, gentamycin, and adding the antibiotic to the medium, thereby obtaining immortalized RPE cells. The viral vector can comprise, for example, genes E6 and/or E7 from HPV 16, and/or telomerase reverse transcriptase genes.

The cells prepared by these methods can retain the in vivo characteristics of non-immortalized RPE cells, such as, for example, cobblestone morphology, polarization, a metabolic pattern typical of non-immortalized RPE cells, the presence of one or more protein markers, and a combination thereof. The RPE cells to be immortalized can be harvested from, e.g., a mouse, a rat or a human.

In another aspect, the invention provides methods of studying an ocular disease. The methods can comprise, for example, providing a culture of immortalized retinal pigmented epithelial cells, modifying the cells to obtain modified immortalized cells, and conducting research on the modified immortalized cells. In some embodiments, the modified immortalized cells exhibit at least one characteristic of an ocular disease, such as, for example, age-related macular degeneration. The modified immortalized cells can also be genetically altered so as to fail to express one or more polypeptides. The polypeptide can be an estrogen receptor such as ERα and/or ERβ. In some embodiments, the methods of studying an ocular disease can comprise isolating retinal pigmented epithelial cells from a cell source to obtain isolated cells, immortalizing the isolated cells to obtain immortalized isolated cells, and conducting research on the immortalized isolated cells. Immortalizing the isolated cells can comprise culturing the isolated cells in a medium under conditions such that the cells are immortalized. Immortalizing the cells can also include, for example, combining the isolated cells with a viral vector comprising a gene that confers resistance to an antibiotic, and adding the antibiotic to the medium, thereby obtaining immortalized isolated cells. The cell source can be, for example, a mouse such as a genetically modified mouse, or a human. In addition, the cell source can be, for example, a model of an ocular disease.

### BRIEF DESCRIPTION OF THE DRAWINGS

FIG. 1. Mouse RPE sheets are viable and isolated cells retain their cobblestone appearance. Comparison of immortalized mouse RPE cells (A) and human RPE cells (B). Mouse RPE cells retain their cobblestone appearance in vitro and have a similar appearance to human RPE cells. C. HPV protein was expressed by all cell lines tested as determined by Western analysis.

FIG. 2. Growth curve of representative RPE cell lines. Cells were plated in medium containing 10% serum and counted following a 24-hour attachment. Wells were counted on days noted on the x axis of the graph. Shown are two representative RPE cell lines, one designated by squares (cell line #14, □) and one by dots (cell line #22, ).

FIG. 3. RPE65 and CRALBP, markers of RPE cells, are expressed in mouse immortalized cells. Western analysis for RPE65 and CRALBP was performed on immortalized, passage 20 RPE cell lysates as described in methods. Lanes 1 and 2: wild type (“wt”) RPE cells no injury; lane 3: estrogen receptor knockout (“ERKO”)β no injury; lane 4: ERKOα no injury; lane 5: wt cells isolated from eyes injured in vivo; lane 6: ERKOα isolated from eyes injured in vivo; lane 7: ERKOβ isolated from eyes injured in vivo; and lane 8: wt cells non transfected no injury. The same blot was stripped and reprobed with an antibody to actin to ensure equal loading of each lane. “In vivo injury” refers to RPE isolated from mice that had been exposed to blue light and high fat diet.

FIG. 4. Immunofluorescent localization of ZO1 (A) and Cytokeratin 8 (B) and Actin (C) are expressed in the pattern characteristic for epithelial cells, on mouse RPE cells in vitro. A: ZO1 positive bands are localized between RPE cells. B and C: Cytokeratin and actin filaments localize to the periphery of the cell. Magnification ×40.

FIG. 5. Ezrin expression. A and B are XY projections of the entire 3D stack of cells. C is a XZ reconstruction of a 10-voxel thick section (Slidebook 4.2) Light Blue DAPI (DNA), red, ezrin. Bars: A-B: 20 μm; C: 10 μm. D. Western blot analysis of three representative cell lines (#1 and #3; wildtype cell lines, #2; ERKOβ cell line) revealed the presence of ezrin protein which increased over time.

FIG. 6. Junctions are present in immortalized mouse RPE cells. Mouse RPE cell lines were grown on coverslips for 10 days and fixed for electron microscopy (“EM”) as described herein. Representative photograph of RPE cell. Each upper inset depicts a single “kiss” tight junction. Scale bar 2 μM.

FIG. 7. ERα (A) and ERβ (B) protein expression in mouse RPE cells is not altered by immortalization. Western analysis of RPE cell lysates as described herein. A. Lanes 1-3 represent three individual cell lines. B. Lanes 1-4 represent four individual cell lines. Rec; recombinant protein. Arrow indicates band of interest, 65 kDa for ERα and 57 kDa for ERβ.

FIG. 8. There is no difference in ER subtype expression between immortalized cells and non-immortalized cell lines. A. Estrogen receptor α. B. Estrogen receptor β. In both (A) and (B), lane 1 depicts non-immortalized cells, and lanes 2-4 depict immortalized cells.

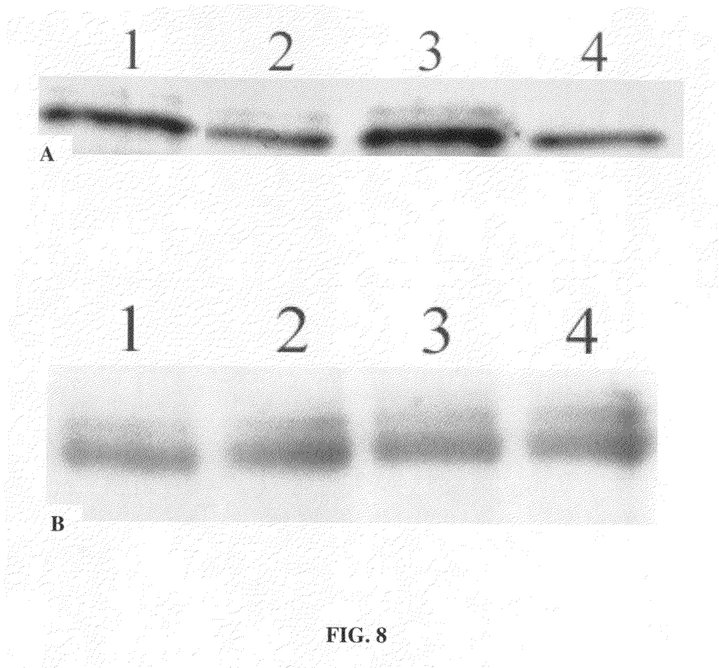

### DETAILED DESCRIPTION

Embodiments of the invention are discussed in detail below. In describing embodiments, specific terminology is employed for the sake of clarity. However, the invention is not intended to be limited to the specific terminology so selected. A person skilled in the relevant art will recognize that other equivalent parts can be employed and other methods developed without parting from the spirit and scope of the invention. All references cited herein are incorporated by reference, in their entirety and for all purposes, as if each had been individually incorporated.

The retinal pigment epithelium is the pigmented cell layer just outside the neurosensory retina. It functions to nourish retinal visual cells, and is also involved in the vitamin A cycle where it isomerizes all trans retinol to 11-cis retinal. The retinal pigment epithelium controls the retinal environment by supplying small molecules such as amino acids, ascorbic acid and D-glucose to retinal cells while maintaining a tight barrier against the entry of other blood-borne substances. The ionic environment is maintained by a delicate transport exchange system.

Models of ocular disease according to the present invention include, for example, immortalized mouse RPE cell lines from C57B1/6 male and female mice, as well as male and female ERKOα and ERKOβ mice. The cell lines prepared according to the present invention retain their cobblestone morphology and are contact inhibited at confluence. Immortalized RPE cells in vitro express in vivo epithelial cell proteins including ZO1, which is expressed at tight junctions, cytokeratin 8 and 18, and actin filaments, which are expressed in a pattern consistent with epithelial cells. Importantly, RPE65 and CRALBP, molecules synthesized by differentiated RPE in vivo, are expressed by all of the cell lines. Expression of these molecules has been shown in human cell line ARPE-19 and the rat cell line RPE-J (Dunn et. al, Exp. Eye Res. 62, 155-69, 1996; Nabi et. al, J Cell Sci 104, 37-49, 1993). Synaptic vesicle protein 2 (“SV2”), a marker of neural cells, as well as rhodopsin, a photoreceptor marker that is generally not found in RPE cells, were not expressed by the cell lines prepared according to the present invention. Furthermore, immortalization of RPE cells using the E6/E7 open reading frames of the HPV16 does not alter any of the in vivo markers or characteristics tested. To study some of the aspects of in vivo pathology, RPE cell lines should ideally maintain their in vivo epithelial properties in vitro. According to the present invention, ezrin, a membrane organizing protein (Hayashi et. al, J. Cell Sci 112 (Pt 8), 1149-58, 1999), can be expressed in the immortalized RPE cell lines in a time dependent manner. Confocal microscopy revealed that ezrin was not fully localized to the apical membrane, suggesting that the cells are not fully differentiated although they express multiple markers found in vivo. EM studies showed that junctions were present in all cell lines tested. The methods of the present invention can be used with cell lines isolated from aging mice. Recent studies have suggested that RPE cells acquire subtle but important age-related changes in phenotype.

According to the present invention, RPE cells that are altered in vivo by oxidant injury or other genetic manipulations exhibit stable changes over multiple passages, for example up to or at least about 32 passages.

ECM components of RPE cells isolated from wildtype littermate control mice have not been altered by immortalization. This is important for future studies utilizing these cell lines to determine therapeutic options to target, for example, the ECM dysregulation that may occur in early dry AMD. Immortalization by the presently claimed methods does not change any of the trimolecular complex components necessary for MMP-2 activation nor collagen type I or type IV mRNA expression. Importantly, the ratio of ERα/ERβ and the ER subtype protein expression also is not altered by immortalization. Other immortalization vectors, for example SV40, alter steroid hormone receptors, including estrogen receptors. Studies on RPE-related physiology and pathophysiology, such as the role of estrogens in controlling ECM turnover in RPE cells, as well as research into the treatment of affected subjects with compounds that target estrogen receptor activation and levels of ECM components, will be greatly advanced by the ability to isolate mouse RPE cell lines.

Immortalized Cells

The invention provides isolated cultures of immortalized retinal pigmented epithelial cells. Each immortalized retinal pigmented epithelial cell comprises one or more genes that confer immortality. As used herein, “immortalized” means not subject to cellular aging processes that might eventually render a cell line unusable for either research or other purposes.

The genes that confer immortality to the retinal pigmented epithelial cells can be introduced into the cells through the use of a viral vector. The vector can comprise the E6/E7 genes from HPV 16. The vector can also comprise telomerase reverse transcriptase (TERT) genes. The TERT genes can be derived from the same species as the cells being immortalized, and are believed to confer immortality by preventing the shortening of the chromosome's telomeres, a process linked to senescence of cells. A pGRN145 hTERT-expressing plasmid can be purchased from ATCC, and introduced into cells by methods known in the art. Immortalized cells can also be obtained by infecting the retinal pigmented epithelial cells with both the HPV E6/E7 genes and the TERT gene.

The retinal pigmented epithelial cells immortalized by the methods disclosed herein can exhibit in vivo characteristics of non-immortalized retinal pigmented epithelial cells. As used herein, “non-immortalized” means subject to cellular aging processes that might eventually render a cell line unusable for either research or other purposes. “Non-immortalized” encompasses 1) cells that have not undergone any step of any immortalization method; 2) cells that have undergone all of the steps of an immortalization method, but for some reason have not become immortalized; and 3) cells that have undergone a modified version of an immortalization method, wherein the modifications result in the method being ineffective at conferring immortality. For example, a cell that has been transfected with a viral vector comprising some, but not all, of the genes necessary for immortalization, and accordingly does not become immortalized, is a non-immortalized cell.

As used herein, “in vivo characteristics” encompasses any trait, whether phenotypic, genotypic or otherwise, and/or any metabolic process that is, or can be, observed in the cell, cell line or organism under consideration. Examples of in vivo characteristics of non-immortalized cells that are exhibited by immortalized RPE cells include, without limitation, cobblestone morphology, polarization, a metabolic pattern typical of non-immortalized RPE cells, and the presence and/or expression of markers characteristic of non-immortalized retinal pigmented epithelial cells. Examples of markers that are characteristic of non-immortalized retinal pigmented epithelial cells include, without limitation, MMP-2, TIMP-2, collagen type IV, ERα, ERβ, RPE65, CRALBP, ZO1, cytokeratin 8, cytokeratin 18, and ER copy number ratio. “ZO1,” or “zona occludens-1,” refers to a marker for epithelial tight junctions. “TIMP” means tissue inhibitor of metalloproteinase. “TIMP-2” can refer to tissue inhibitor of metalloproteinase-2. “TIMP-2” can also mean TIMP metallopeptidase inhibitor 2. “RPE65” refers to retinal pigment epithelium-specific protein 65 kDa. In some embodiments, these markers are expressed or otherwise present in substantially the same amounts as in non-immortalized retinal pigmented epithelial cells. RPE cells generally express RPE65 and CRALBP. These markers are also found in other cell types.

The RPE cells for immortalization can be harvested from a subject. As used herein, “harvest” refers to obtaining cells from their in vivo environment in a subject by any means known to those of skill in the art, including those disclosed herein. As used herein, a “subject” can be any animal whose cells are capable of being immortalized by the methods of the present invention, for example, a mouse, a rat or a human. In some embodiments, the cells exhibit one or more characteristics of an ocular disease. Such cells can be useful in studying, e.g., the onset and/or progression of the ocular disease, as well as possible treatments. The ocular disease can be any ocular disease whose characteristics can be retained and exhibited by the RPE cells after immortalization, such as, for example, AMD, proliferative vitreoretinopathy, Menkes and Wilson diseases, vitelliform macular dystrophy, acute retinal pigment epithelitis, RP hereditary macular degeneration and myopic degeneration. The ocular disease, or the one or more characteristics exhibited by the RPE cells, can be present in the cells harvested from the subject. The characteristics of the ocular disease can be found to exist in the subject, or the cells or subject can have been modified prior to harvesting by any means known in the art, for example genetically or by administration of drugs that bring about the modification. In addition, the cells can be modified after harvesting by any means known in the art such that they exhibit one or more characteristics of the ocular disease. Immortalization can occur before, simultaneously with, or after the cells are modified so as to exhibit the ocular disease, or the one or more characteristics of the ocular disease.

Methods of Preparing Immortalized Cells

In another aspect, the invention provides methods of preparing an immortalized RPE cell line. In some embodiments, the methods comprise harvesting the RPE cells from a subject, and culturing the RPE cells in a medium under conditions such that the cells are immortalized. As used herein, “medium” encompasses any substance on which RPE cells can be grown or sustained. Immortalization may be achieved, e.g., by infecting cells with a vector, such as with a recombinant plasmid, a recombinant virus or a retrovirus, e.g. a recombinant retroviral vector carrying the E6 and/or E7 genes of an HPV virus, for example HPV 16. In some embodiments, the recombinant vector harbors TERT genes, which can be derived from the same species as the cells being immortalized. The methods can also comprise infection with both the HPV E6/E7 genes and TERT genes. The RPE cells immortalized according to the methods of the invention retain the in vivo characteristics of RPE cells. The cells can exhibit one or more characteristics of an ocular disease, which can be present in the RPE cells harvested from the subject or induced at a later time, e.g., before or after immortalization.

Selection is made for cells positively immortalized, e.g. by culturing the cells for several passages or by testing the cells for genes of the vector used for the immortalization, such as for genes from the HPV virus. This may be achieved by detecting the expression of the respective genes by means of antibodies or may involve an analysis via PCR-techniques. Expression of the telomerase reverse transcriptase genes may be detected by measuring the telomerase activity determined by applying the Telomerase Repeat Amplification Protocol (TRAP).

In some embodiments, cells can be exposed to replication-deficient retrovirus containing HPV 16 E6/E7 DNA and also a gene that confers resistance to an antibiotic. The antibiotic can be, without limitation, gentamycin. For example, this exposure can be carried out by introducing a replication-deficient retrovirus containing HPV 16 E6/E7 DNA with a gentamycin resistant gene into a well containing the cells to be immortalized in the presence of polybrene. Later, the medium can be changed, and gentamycin can be added for, e.g., several days to select those cells which have incorporated the virus. See, e.g., Fontijn, R., Hop, C., Brinkman, H. J., Slater, R., Westerveld, A., van Mourik, J. A. and Pannekoek, H. (1995). “Maintenance of Vascular Endothelial Cell-Specific Properties after Immortalization with an Amphotrophic Replication-Deficient Retrovirus Containing Human Papilloma Virus 16 E6/E7 DNA.” Exp. Cell Res. 216, 199-207. The particular steps and/or parameters in this method of selecting immortalized RPE cells can be varied, so long as the method results in the selection of RPE cells that have successfully incorporated the viral vector conferring immortality.

In some embodiments, the method of preparing an immortalized retinal pigmented epithelial cell line can comprise, for example, harvesting retinal pigmented epithelial cells from a subject; culturing the cells in a medium; combining the cells with a viral vector comprising a gene that confers resistance to an antibiotic agent, for example gentamycin; and selecting the cells that incorporated the vector by adding the antibiotic agent to the medium, thereby destroying the cells that did not incorporate the vector. Suitable vectors for use according to the invention can comprise, for example, human papilloma virus 16 E6/E7 DNA, or the TERT gene, for example derived from the same species as the cells being immortalized. The subject can be any animal whose cells are capable of being immortalized by the methods of the present invention, for example, a mouse or a human. In another aspect, the invention relates to the isolated culture of immortalized retinal pigmented epithelial cells obtained by the methods described herein.

Table 1 presents a listing of mouse cell lines and their characteristics, prepared according to the methods disclosed herein.

TABLE 1
Mouse cell lines

| Cell Line | Alt. Cell Line | Exposure | | |
|---|---|---|---|---|
| No. | No. | to Injury | Description | Gender |
| 1 | 2178 | L HFD | ERKO β | F |
| 2 | 2180 | L HFD | ERKO β | F |
| 3 | 2181 | L HFD | ERKO β | F |
| 4 | 2183 | L HFD | ERKO β | F |
| 5 | 2185 | L HFD | ERKO α | F |
| 6 | 2188 | L HFD | ERKO α | F |
| 7 | 2193 | L HFD | ERKO β wt | F |
| 8 | 2194 | L HFD | ERKO β wt | F |
| 9 | 2195 | L HFD | ERKO β wt | F |
| 10 | 2196 | L HFD | ERLO β wt | F |
| 11 | 2198 | L HFD | ERKO α wt | F |
| 12 | 2211 | L HFD | ERKO α wt | F |
| 13 | 2212 | L HFD | ERKO α wt | F |
| 14 | 2213 | L HFD | ERKO α wt | F |
| 15 | 2214 | L HFD | ERKO β | F |
| 16 | 2215 | L HFD | ERKO β | F |
| 17 | 2216 | L HFD | ERKO β | F |
| 18 | 2217 | Control | ERKO β | F |
| 19 | 2218 | Control | ERKO β | F |
| 20 | 2219 | Control | ERKO β wt | F |
| 21 | 2220 | Control | ERKO α | F |
| 22 | 2221 | Control | ERKO α wt | F |
| 23 | 2222 | Control | ERKO α | M |
| 24 | 2223 | Control | ERKO α | M |
| 25 | 2224 | Control | ERKO β | M |
| 26 | 2225 | Control | ERKO β wt | M |
| 27 | 2227 | L HFD | ERKO β | F |
| 28 | 2228 | L HFD | ERKO β wt | F |
| 29 | 2229 | L HFD | ERKO β wt | F |
| 30 | 2230 | L HFD | ERKO α | F |
| 31 | 2231 | L HFD | ERKO α | F |
| 32 | B6 | 3 months | transfected | F |
| 33 | B6 | 10 months | transfected | F |

Notes:
Wt = wildtype
L = light
HFD = high fact diet
ERKO = estrogen receptor knockout
ERKOα wt = littermate control mouse from the ERKO colony

Methods of Studying Ocular Disease

In still another aspect, the invention relates to methods of studying ocular disease. In some embodiments, the methods comprise obtaining modified RPE cells; immortalizing the modified RPE cells, for example as obtained by any of the methods disclosed herein; and conducting research on the resulting modified immortalized cells. Modifying the cells can result in cells that express one or more characteristics of an ocular disease. This modification can be done by any method known in the art, including, without limitation, transfection, knockdown and/or knockout. For example, modifying the cells can involve genetically altering the cells so as to fail to express a polypeptide, for example an estrogen receptor such as ERα or ERβ. Such a modification can allow an investigator to study the polypeptide's role in the etiology of the disease. Modifying the cells can involve multiple modifications. For example, the same cell line can be modified so as to express one or more characteristics of an ocular disease and also to fail to express a polypeptide such as ERα and/or ERβ.

The resulting modified immortalized cells can be used to study an ocular disease. The research conducted on the modified immortalized cells can involve any methods now known or hereafter developed to study the cause, onset, progression, treatment or other aspect of the disease.

In some embodiments, the method of studying an ocular disease can comprise one or more of the following steps: isolating RPE cells from a model of an ocular disease, such as, for example, genetically modified mice; immortalizing the RPE cells by one or more of the methods of the present invention; and conducting research on the resulting immortalized isolated RPE cells. “Model of an ocular disease” encompasses any cell source, such as a human, an animal such as a mouse, or a cell culture, in which the cells express at least one symptom or trait of an ocular disease. The subject from which the RPE cells are harvested can have been genetically modified so as to express one or more characteristics of an ocular disease, or to fail to express a polypeptide (e.g., ERα, ERβ, or both), prior to harvesting of the RPE cells for immortalization.

As used herein, “conducting research” may comprise any research or investigational method, whether currently known or hereafter developed, whether on a disease or on another aspect of the cell line. If the research relates to a disease, it can relate to any such disease, including those described herein. “Conducting research” can also encompass studying the progression of the disease and its impact on other organ systems, as well as the overall functioning of the organism.

In a particular embodiment, “conducting research” can involve, for example, screening of test compounds for efficacy in treating an ocular disease such as AMD. Such treatment can comprise, for example, changing the cellular characteristics in such a way that the causes and/or symptoms of the ocular disease are reduced or eliminated, or the progression of the disease is slowed. For example, the screening method may comprise contacting immortalized cells (e.g. isolated immortalized RPE cells) with a test compound, and measuring the effect of that test compound on one or more characteristics (e.g. the amount of expression of a particular protein) associated with a disease (e.g. age-related macular degeneration), comparing the result to a control condition (e.g. no treatment, or treatment with a control compound), wherein a decrease or increase in the characteristic is indicative of a compound that is a candidate for therapeutic testing or use.

Additional objects, advantages, and novel features of the present invention will become apparent to one of ordinary skill in the art upon consideration of the following examples, which are not intended to limit the scope of the invention in any way.

### EXAMPLES

### Example 1

### Preparation of Immortalized Retinal Pigmented Epithelial Cells

Methods:

Isolation of RPE sheets from mice: Intact eyes were removed quickly from 8 week and 16 month old C57B1/6 mice (Jackson Laboratories, Bar Harbor, Me.) and 16 month old male and female ERKO mice following anesthesia. The eyes were washed twice in Dulbecco's modified eagle's medium (“DMEM”) containing high glucose, followed by an enzymatic digestion in 2% Dispase in DMEM for 45 minutes at 37° C. Eyes were washed two times in growth medium (“GM”) consisting of DMEM high glucose, 10% fetal bovine serum (“FBS”), 1% penicillin/streptomycin, 2.5 mM/L-Glutamine and 1% non-essential amino acids. After washing, the eyes were transferred into fresh GM medium for dissection.

Using microdissection scissors and an upright dissection microscope, a circular incision was made around the ora serrata of each eye. The posterior eyecup containing the neural retina and the lens were placed in fresh GM medium and incubated for 20 minutes at 37° C. in 10% CO₂ incubator to facilitate separation of the RPE sheets from the neural retina.

After removal of the RPE sheets from the neural retina, intact sheets of RPE cells were peeled and collected in an eppendorf tube. RPE were centrifuged at 1500 rpm for 5 minutes and resuspended in GM medium. The cell suspension (0.5 ml) was added to the upper chamber of a 12-well plate, containing polycarbonate transwell inserts with a 3.0 μm pore diameter. GM medium was added to the lower well (1 ml). Cells were cultured at 37° C. in 10% CO₂ for 10 days, with a change of medium (GM) every other day. After 10 days the cells were washed with versene for 3 minutes and then trypsinized for 4 minutes to detach the cells from the insert. The cells were collected in a tube, centrifuged at 1000 rpm for 5 minutes and resuspended in DMEM/F:12 10% FBS PEN/strep, 1-glutamine, sodium bicarbonate. The cells were plated in 24-well plates until they reached confluence, at which time they were trypsinized and grown in a larger flask.

Viability Assay: Isolated sheets of RPE cells were tested for viability using a viability kit from Molecular Probes, Inc., Eugene, Oreg. The live/dead viability kit is a two-color assay which identifies live versus dead cells on the basis of membrane integrity and esterase activity. The combine solution included in the kit was added to the tissue for 45 minutes at room temperature immediately after isolation. Using fluorescent microscopy, viability was assessed based on color, live cells in green and dead cells in red.

Immortalization: Cells were plated in 24-well plates and allowed to come to semi-confluence. Media was removed and a replication-deficient retrovirus containing human papilloma virus 16 E6/E7 DNA with a gentamycin resistant gene, was added to each well in the presence of polybrene (4 μg/ml). Twenty-four hours later the medium was changed and gentamycin (800 μg/ml) added for 3 days to select those cells which have incorporated the virus. Fontijn, R., et. al, Exp. Cell Res. 216, 199-207.

Following treatment the surviving cells were passaged and characterized. A portion of the same cell culture was grown in a sister well to confluence, but was not subjected to immortalization and served as controls for the characterization experiments. An antibody against HPV 16 E6 was used to assure the presence of HPV. Cell lines are designated numerically, e.g., 1-32. For example, cell lines can be designated with a set of leading numbers included ahead of the cell line designation, such as “22.”

Growth Curves: Cells were plated at equal density in medium containing 10% FBS in 24-well plates. Twenty-four hours later, cells were counted to determine plating efficiency. Cell number was assessed on days 1-5 and continued until day 15 with a Coulter Z1 cell counter (Beckman Coulter, Hialeah, Fla.).

Electron Microscopy: RPE cells were grown on German Glass cover slips (Carolina Biological, Inc., Burlington, N.C.) to confluence. The monolayers were fixed in 2% glutaraldehyde/100 mM Sucrose/0.05M PO₄ for 1 hour. The samples were further dehydrated through graded ethanol, embedded in Epon-812 and polymerized overnight at 64° C. Thick and ultrathin sections (0.7-1.0 μm) were cut on a microtome (MT-2; Porter Blum, Hatfield, Pa.). Thick sections were stained with toluidine blue and examined by light microscopy. Ultrathin sections were stained with 4% uranyl acetate and lead citrate and examined with a transmission electron microscope (model CX-100; JEOL, Tokyo, Japan).

Immunohistochemistry: RPE cells were plated in either 24-well plates or 8-well chamber slides for 7-10 days prior to fixation. Cells were washed in phosphate buffered saline (“PBS”) and fixed with either 4% paraformaldehyde (RPE65, cytokeratin 8 and 18, ZO1 and actin), or 10% TCA (Ezrin) for 10 minutes, depending on the antibody staining to be performed. This was followed by three washes with PBS for 10 minutes to remove excess fixative solution, and blocking for 1 hour at room temperature with a solution containing 5% bovine serum albumin (“BSA”) and 1% Triton in PBS. The blocking solution was removed, the slide was washed in PBS for 5 minutes and one of the following primary antibodies was added: RPE-65 (Dr. Michael Redmond), used at 1:1000; cytokeratin 8 and 18 (Dr. Boris Stanzel), used at 1:2000 and 1:1000, respectively; and ZO-1 (Invitrogen cat#33-9100), at 1 μg/ml. Cells were also stained with ezrin monoclonal antibody (Covance, 5 μg/ml). Ezrin is an actin-binding protein that is crucial for morphogenesis of apical microvilli and basolateral infoldings of RPE cells. Cells were incubated overnight with primary antibodies or exposed to the antibody against ZO-1 or rhodamine phallodoin for 1 hour, washed and mounted. Following an overnight incubation with the primary antibody, the cells were rinsed three times with PBS for 10 minutes, and the secondary antibody (Alexa Fluor 546 goat anti-rabbit or Alexa Fluor 546 goat anti-mouse; 4 mg/ml) was added for 2 hours. The second antibody allows for visualization since it is fluorescent. It binds to the first antibody. The cells were then washed five to six times in PBS, mounted and examined with a Leica microscope.

Western Blot analysis: Western analysis was performed according to methods known to those of ordinary skill in the art. See, e.g., Elliot, S., Catanuto, P., Stetler-Stevenson, W. and Cousins, S. W. (2006). “Retinal Pigment Epithelium Protection From Oxidant-mediated Loss of MMP-2 Activation Requires Both MMP-14 and TIMP-2.” Invest Opthalmol. Vis. Sci. 47, 1697-702. Briefly, cells were plated in T75 flasks and allowed to grow to confluence. The cells were maintained at confluence for a period of one week, at which time they were collected into lysis buffer containing a protease inhibitor cocktail. Protein was extracted and a protein assay performed to determine loading amounts. Fifty μg of protein was electrophoresed on a 10% polyacrylamide gel for RPE65 (Millipore, Billerica, Mass.) and cellular retinaldehyde-binding protein (CRALBP, Abcam Inc., Cambridge, Mass. For ERα 40 μg of protein was loaded and for ERβ 100 μg of protein was immunoprecipitated, according to methods known to those of ordinary skill in the art. See, e.g., Karl M, Potier M, Schulman I H, Rivera A, Werner H, Formoni A, Elliot S J. “Autocrine Activation of the Local IGF-I System Is Up-Regulated by Estrogen Receptor (ER)-Independent Estrogen Actions and Accounts for Decreased ER Expression in Type 2 Diabetic Mesangial Cells.” Endocrinology, 146: 889-900, 2005. All western blots were exposed to actin to normalize for loading variability. In addition, cells were also tested for the presence of SV2, to rule out contamination with neural cells, as well as of rhodopsin and protein kinase C-alpha (“PKCα”), markers of photoreceptors and bipolar rods, respectively.

Real-time RT-PCR: Real-time PCR was performed according to methods known to those of ordinary skill in the art on isolated RPE cells before and after immortalization. Elliot, S. et al., Invest Opthalmol. Vis. Sci. 47, 1697-702. Briefly, RT-PCR reactions were performed using the TaqMan One Step RT-PCR Master Mix reagent kit and the ABI Prism 7700 sequence detection system (Perkin Elmer Applied Biosystems) in a total volume of 50 μl of reaction mixture. A TaqMan ribosomal probe RNA control reagent kit was used to detect the 18S ribosomal RNA gene, which represented an endogenous control. Baseline mRNA expression of RPE65, MMP-14, MMP-2, TIMP-2, collagen type I and type IV and ERα and ERβ was determined. Each sample was normalized to the 18S transcript content.

Flow cytometry: Cells were analyzed by flow cytometry using a FACScan (Beckton Dickinson). Briefly, cells were trypsinized, and fixed in 70% ethanol overnight. Ethanol was removed by multiple PBS washes and 50 μg/ml of propidium iodide was added. Cells were measured by width and area and plotted by area. Following analysis, the amount of DNA, which reflects the number of cells or the stage of the cell cycle, was quantified by incorporation of nucleic acid stains (propidium iodide).

Results and Summary:

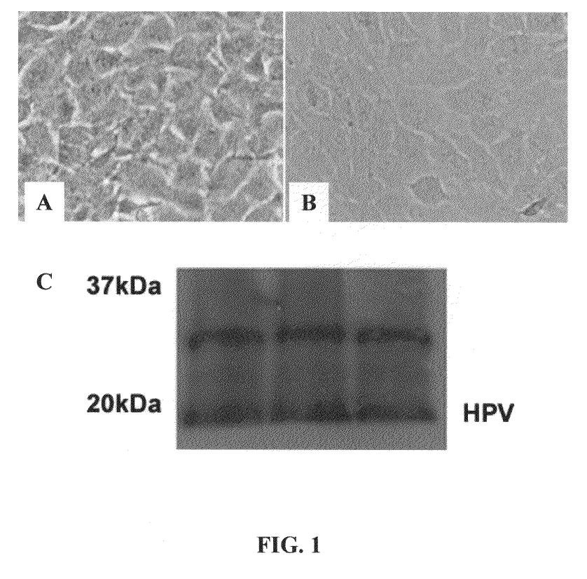

Viability: The majority of the cells on the isolated sheets were viable as assessed by the live/dead assay. The cell lines were propagated to obtain a bank of frozen cell lines. In addition, cells have been frozen and thawed multiple times without noticeable changes in morphology. Immortalization of mouse RPE cell lines had no effect on morphology of the cell lines. They retained their cobblestone appearance and were contact inhibited. (See FIG. 1A (mouse immortalized RPE cells) and B (human RPE cells)). Spontaneous transformation of the cell lines was ruled out by the presence of HPV protein, which was expressed by all cell lines tested as determined by Western analysis (FIG. 1C).

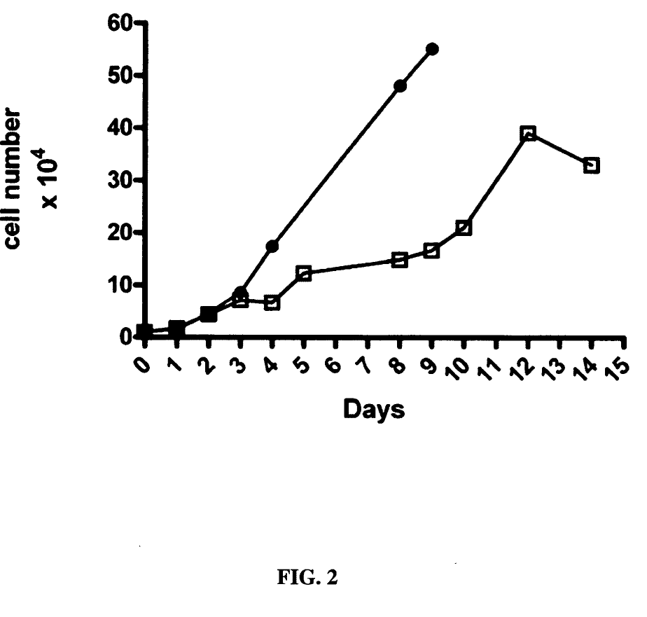

Growth Curves: The initial doubling time for most cell lines was 16-24 hours as has been described for ARPE-19 cells (Dunn et. al, 1996). As the cells approached confluency, cell number increased (FIG. 2) at a slower rate, although there was variability between cell lines. The cells were maintained in a monolayer for 7-14 days before peeling off in sheets.

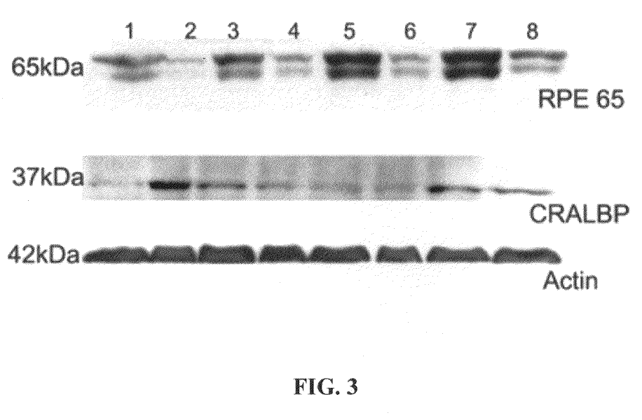

Specific Markers of RPE cells: To confirm the presence of in vivo RPE markers, we performed RT-PCR and western analysis of cultured cells (FIG. 3). The mRNA expression of RPE65 was consistent across individual cell lines (Table 2). The protein expression of RPE65 and CRALBP, another marker of RPE cells expressed in vivo, was evident in non-transformed and immortalized RPE cells (FIG. 3—Lanes 1 and 2: wildtype (“wt”) RPE cells, no injury; lane 3: ERKOβ, no injury; lane 4: ERKOα, no injury; lane 5: wt cells isolated from eyes injured in vivo lane 6: ERKOα isolated from eyes injured in vivo; lane 7: ERKOβ isolated from eyes injured in vivo; lane 8: wt cells, non-transfected, no injury). The same blot was stripped and reprobed with an antibody to actin to ensure equal loading of each lane. Expression of these molecules has been shown on human ARPE-19 cell lines (Dunn et. al, Exp. Eye Res. 62, 155-69, 1996). An interesting finding was the differential regulation of RPE65 in the absence of ERβ (lanes 3 and 7) even in the absence of injury in vivo (lane 3). In addition, SV2 expression, a neural cell marker, as well as expression of PKCα and rhodopsin were negative.

TABLE 2
mRNA expression of RPE65 in mouse cell lines

| Cell Line | Description | RPE65/18s |
|---|---|---|
| 15 | ERKOβ | 1.75 |
| 29 | wtERKOβ | 1.69 |
| 12 | wtERKOα | 1.78 |
| 31 | ERKOα | 1.73 |
| 22 | ERKOα | 1.9 |

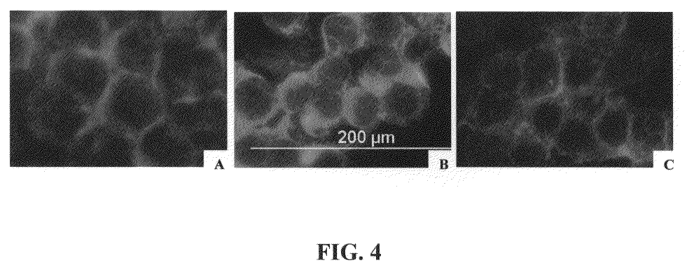

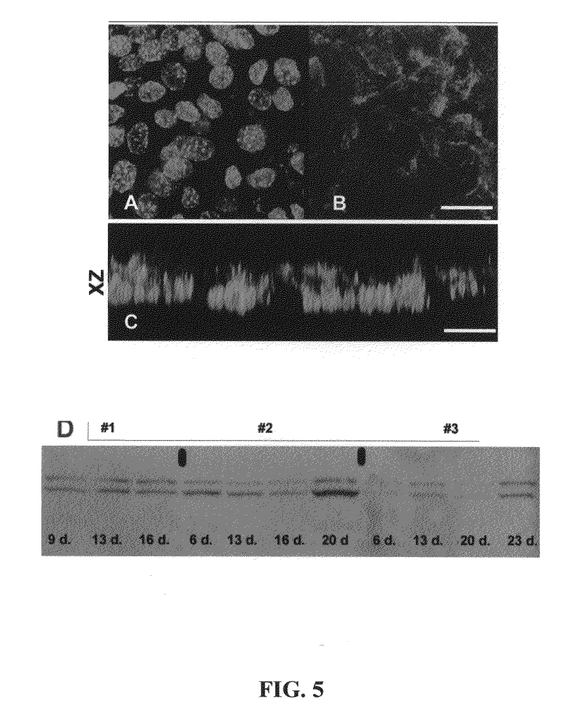

Markers of epithelial cell junctions and polarity: ZO-1 (FIG. 4A), cytokeratin 8 and cytokeratin 18 were also expressed on RPE sheets, and isolated cells in culture at a level similar to that observed in rat RPE-J cells (Nabi et al., J Cell Sci 104, 37-49, 1993). ZO1, cytokeratin 8 and actin are localized and expressed in the pattern characteristic for epithelial cells, on mouse RPE cells in vitro. ZO1 positive bands are localized between RPE cells. Importantly, all of these markers were present up to at least 32 passages after immortalization. Actin filaments were distributed predominantly about the cell periphery as described for other epithelial cells (FIG. 4B). Ezrin immunostaining revealed partial localization to the apical surface on RPE cell lines by Z-scans performed through the monolayer. Ezrin localization at the apical surface was also shown using confocal imaging (FIG. 5A). Mouse RPE cell lines expressed ezrin in a time dependent manner (FIG. 5B).

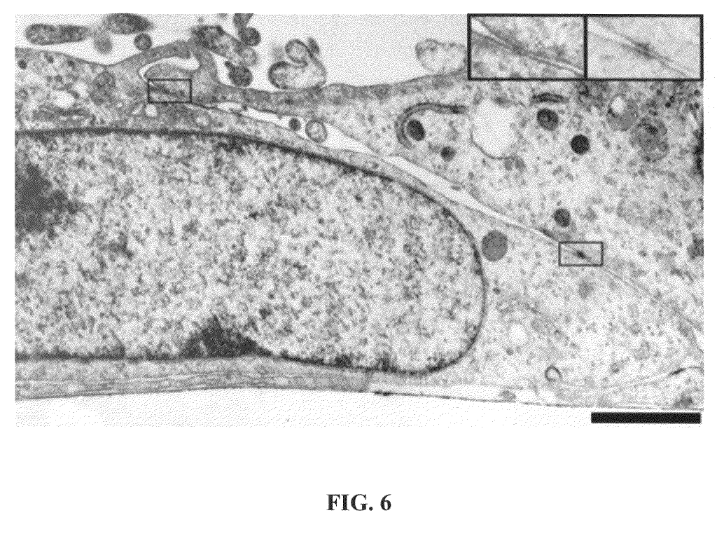

Electron Microscopy: EM revealed the presence of junctions (FIG. 6) in all RPE cell lines studied. This is in agreement with ARPE-19 cells grown on matrigel coated transwell filters, which possess junctional complexes (Dunn et. al, Exp. Eye Res. 62, 155-69, 1996).

PCR: It was found that mRNA expression for MMP-2, TIMP-2, MMP-14, collagen type IV and ERα/ERβ ratio did not differ between immortalized and non-immortalized wildtype cultures of RPE cells (Table 3). Comparisons were only made on wildtype cell lines for this part of the characterization because we found a difference in expression of extracellular matrix molecules between ERKO cell lines and wildtype littermates as a consequence of the presence or absence of ERα or ERβ (Elliot et. al, Exp. Eye Res. 60, 653-60, 2008).

TABLE 3
RPE mRNA expression before and after immortalization
(representative wildtype cell lines)

| | RPE | ERα/ERβ ratio | | | | Collagen |
|---|---|---|---|---|---|---|
| Ratio molecule/18s | 65 | (copy number) | MMP-2 | TIMP-2 | MMP-14 | IV |
| Non-immortalized cells | 1.87 | 2.6 | 84 | 123 | 6.3 ± 1.5 | 159 |
| Immortalized cells | 1.9 | 2.6 | 91 | 127 | 5.9 ± 1.3 | 152 |

Flow Cytometry: We found no difference between immortalized and non-immortalized cell lines in the number of cells in the respective stages of the cell cycle. 55.3±11% of immortalized cells were in G1, 13±4.5% in S, and 27±13% in G2. Non-immortalized cells had 53±15.4% in G1, 16.7±5.7% in S, and 23.5±6.9% in G2 (n=3).

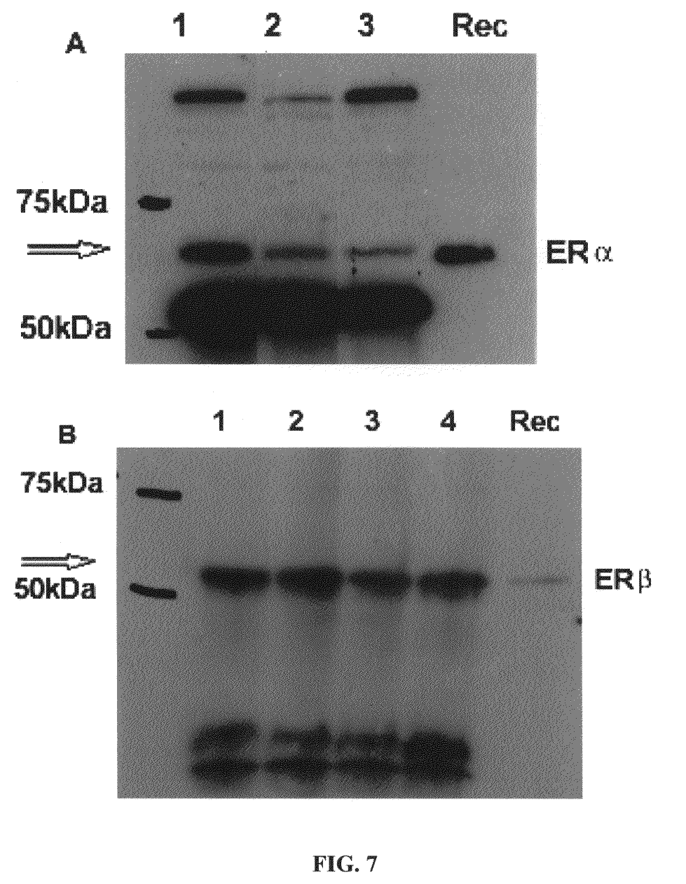

Western blot analysis of ER subtypes: Western analysis revealed the presence of both ER subtypes in wildtype cells (FIGS. 7A and B), as has previously been shown in human ARPE-19 cells and primary human RPE cells (Marin-Castano et. al, Invest Opthalmol. Vis. Sci. 44, 50-9, 2003).

The embodiments illustrated and discussed in this specification are intended only to teach those skilled in the art the best way known to the inventors to make and use the invention. Nothing in this specification should be construed as limiting the scope of the present invention. All examples presented are representative and non-limiting. The above-described embodiments of the invention may be modified or varied without departing from the invention, as appreciated by those skilled in the art in light of the above teachings. It is therefore to be understood that, within the scope of the claims and their equivalents, the invention may be practiced otherwise than as specifically described.

1. An isolated culture of immortalized retinal pigmented epithelial cells, wherein each cell comprises one or more genes that confer immortality.

2. The culture of claim 1, wherein the one or more genes are selected from the group consisting of HPV 16 E6, HPV 16 E7, and combinations thereof.

3. The culture of claim 1, wherein, the one or more genes comprise the telomerase reverse transcriptase genes.

4. The culture of claim 1, wherein the immortalized cells retain one or more of the in vivo characteristics of non-immortalized retinal pigmented epithelial cells.

5. The culture of claim 4, wherein the one or more in vivo characteristics are selected from the group consisting of cobblestone morphology, polarization, the presence of one or more protein markers, a metabolic pattern typical of non-immortalized retinal pigmented epithelial cells, and a combination thereof.

6. The culture of claim 5, wherein one or more protein markers are selected from the group consisting of MMP-2, TIMP-2, collagen type IV, ERα, ERβ, RPE65, ZO1, cytokeratin 8, cytokeratin 18, ER copy number ratio, and combinations thereof.

7. The culture of claim 6, where the one or more protein markers are expressed in substantially the same amounts as in non-immortalized retinal pigmented epithelial cells.

8. The culture of claim 1, wherein the immortalized cells were harvested from a mouse prior to immortalization.

9. The culture of claim 1, wherein the immortalized cells were harvested from a human prior to immortalization.

10. The culture of claim 1, wherein the immortalized cells exhibit one or more characteristics of an ocular disease.

11. The culture of claim 10, wherein the ocular disease is age-related macular degeneration.

12. A method of preparing an immortalized retinal pigmented epithelial cell line, comprising:
harvesting retinal pigmented epithelial cells from a subject; and
culturing the retinal pigmented epithelial cells in a medium under conditions such that the cells are immortalized.

13. The method of claim 12, wherein the immortalized retinal pigmented epithelial cells retain the in vivo characteristics of non-immortalized retinal pigmented epithelial cells.

14. The method of claim 13, wherein the one or more in vivo characteristics are selected from the group consisting of cobblestone morphology, polarization, the presence of one or more protein markers, a metabolic pattern typical of retinal pigmented epithelial cells, and a combination thereof.

15. The method of claim 12, wherein the subject is a mouse.

16. The method of claim 12, wherein the subject is a human.

17. The method of claim 12, that comprises:
combining the retinal pigmented epithelial cells with a viral vector comprising a gene that confers resistance to an antibiotic; and
adding the antibiotic to the medium;
thereby obtaining immortalized retinal pigmented epithelial cells.

18. The method of claim 17, wherein the viral vector comprises human papilloma virus 16 E6/E7 DNA.

19. The method of claim 17, wherein the antibiotic is gentamycin.

20. The method of claim 12, wherein the immortalized cells exhibit one or more characteristics of an ocular disease.

21. The method of claim 20, wherein the ocular disease is age-related macular degeneration.

22. The method of claim 20, wherein the immortalized cells exhibited the one or more characteristics of an ocular disease prior to harvesting from the subject.

23. The method of claim 20, wherein the immortalized cells were modified so as to exhibit the one or more characteristics of an ocular disease after harvesting and before immortalization.

24. The method of claim 20, wherein the immortalized cells were modified so as to exhibit the one or more characteristics of an ocular disease after immortalization.

25. A culture of immortalized retinal pigmented epithelial cells prepared according to the method of claim 12.

26. A method of studying an ocular disease, comprising:
harvesting retinal pigmented epithelial cells from a cell source to obtain isolated cells;
immortalizing the isolated cells to obtain immortalized isolated cells; and
conducting research on the immortalized isolated cells.

27. The method of claim 26, wherein conducting research comprises screening of test compounds for efficacy in treating an ocular disease.
